(19) United States
(12) Patent Application Publication
(10) Pub. No.: US 2016/0066846 A1
(43) Pub. Date: Mar. 10, 2016

(54) BIOLOGICAL INTERFACE WITH MAGNETIC SENSORS

(71) Applicants: Cora Scott, Malibu, CA (US); William C. Scott, Malibu, CA (US)

(72) Inventors: Cora Scott, Malibu, CA (US); William C. Scott, Malibu, CA (US)

(21) Appl. No.: 14/478,722

(22) Filed: Sep. 5, 2014

(51) Int. Cl. A61B 5/00; A61B 5/0478

(52) U.S. Cl. CPC A61B 5/683; A61B 5/0478; A61B 5/6816; A61B 5/6826; A61B 2562/125

(57) ABSTRACT

Exemplary embodiments of a method and apparatus are provided where a biological interface having magnets can be used to measure electrical activity. A biological interface is provided having a first magnetic attachment having a first magnet with a first polarity, a second magnetic attachment having a second magnet with a second polarity opposite to the first polarity, and a sensor attached to the second magnetic attachment for recording electrical activity. The sensor can be an electrode having a first surface and an opposite second surface, where the first surface is attached to the second magnetic attachment.

### FIELD OF THE DISCLOSURE

The present disclosure relates to exemplary embodiments of a biological interface, and more particularly, to exemplary embodiments of a biological interface having magnetic sensors.

### BACKGROUND INFORMATION

Sensors having electrodes can be used to measure various biological parameters. They are placed on a thin part of a patient's body, such as a finger or an ear lobe. Various biological parameters can be measured, such as brain wave activity, heart rate, skin conductance, temperature, blood oxygen, blood carbon dioxide and muscle tension.

As shown in FIG. 1, which is an illustration of a traditional sensor, sensors 10 and 20 are placed on opposite sides of a finger or ear lobe. A doctor or operator will open the sensors 10 and 20 by applying tension (e.g., by using their fingers) on the grips 40 and 50, which apply tension and rotate the wires 45 and 55 attached to sensors 10 and 20, respectively, around the coil 30 to open the sensors 10 and 20. The doctor or operator can then release the tension applied and close the sensors 10 and 20 on a user's ear lobe or finger to measure the various biological parameters through wire 60.

However, these traditional sensors typically bend and fall off a user's earlobe or finger, as the coil will typically wear out and deteriorate over time. The sensors do not stay in place, fall off, and create unnecessary technical support issues and additional expense (e.g., to repair or replace the device). It would be useful for doctors/patients to be able to use a device that is placed easily and is reusable without wearing out.

### SUMMARY OF EXEMPLARY EMBODIMENTS OF THE DISCLOSURE

At least some of the above described problems can be addressed by exemplary embodiments of the methods and apparatuses according to the present disclosure. For example, using such exemplary embodiments, it is possible to provide an apparatus that can be configured to be used multiple times without wearing out, and that can be easily placed on a user's ear lobe or finger.

The present disclosure describes exemplary embodiments of methods and apparatuses that can provide a biological interface. In some exemplary embodiments, a biological interface can be provided comprising a first magnetic attachment configured to be placed on a first portion of an extremity of a body, the first magnetic attachment having a first magnet with a first polarity, a second magnetic attachment configured to be placed on a second portion opposite the first portion of the extremity of the body, the second magnetic attachment having a second magnet with a second polarity opposite to the first polarity, and a sensor attached to the second magnetic attachment.

The first magnetic attachment can comprise a base configured to hold the first magnet within the base of the first magnetic attachment, wherein a bottom of the base exposes a portion of the first magnet. The biological interface can further comprise a cone shaped portion attached to the base at a first end and a dome at a second end. The second magnetic attachment can comprise a base configured to hold the second magnet within the base of the second magnetic attachment, wherein a bottom of the base exposes a portion of the second magnet. The biological interface can further comprise a cone shaped portion attached to the base at a first end and a dome at a second end.

In some exemplary embodiments, a body of the magnetic attachments can be made of plastic. The biological interface can be used for electroencephalography. The sensor can comprise an electrode. The electrode can have a first surface and a second surface opposite the first surface, and the first surface of the electrode can be attached to the second magnetic attachment. The sensor can be glued to the second magnetic attachment. The extremity can be a finger or ear lobe.

In some exemplary embodiments, a method of manufacturing a biological interface can be provided, comprising providing a first magnet having a first polarity within a first magnetic attachment, providing a second magnet having a second polarity opposite the first polarity within a second magnetic attachment, and attaching a sensor to the second magnetic attachment for recording electrical activity. The sensor can comprise an electrode. The electrode can have a first surface and a second surface opposite the first surface, and the first surface of the electrode is attached to the second magnetic attachment.

The method can further comprise abrading the first surface of the electrode and a base of the second magnetic attachment before attaching the sensor and the second magnetic attachment. The attaching can comprise gluing the first surface of the electrode to a base of the second magnetic attachment. The first magnetic attachment can comprise a base configured to hold the first magnet within the base of the first magnetic attachment, wherein a bottom of the base exposes a portion of the first magnet. The second magnetic attachment can comprise a base configured to hold the second magnet within the base of the second magnetic attachment, wherein a bottom of the base exposes a portion of the second magnet.

These and other objects, features and advantages of the present disclosure will become apparent upon reading the following detailed description of embodiments of the present disclosure, when taken in conjunction with the appended claims.

### BRIEF DESCRIPTION OF THE DRAWINGS

The foregoing and other exemplary objects of the present disclosure will be apparent upon consideration of the following detailed description, taken in conjunction with the accompanying exemplary drawings and claims, in which like reference characters refer to like parts throughout, and in which:

FIG. 1 is an illustration of a traditional sensor;

FIG. 2 is an illustration of a biological interface having magnetic attachments and a sensor according to an exemplary embodiment of the present disclosure; and

FIG. 3 is an illustration of a biological interface having magnetic attachments combined with a sensor according to an exemplary embodiment of the present disclosure.

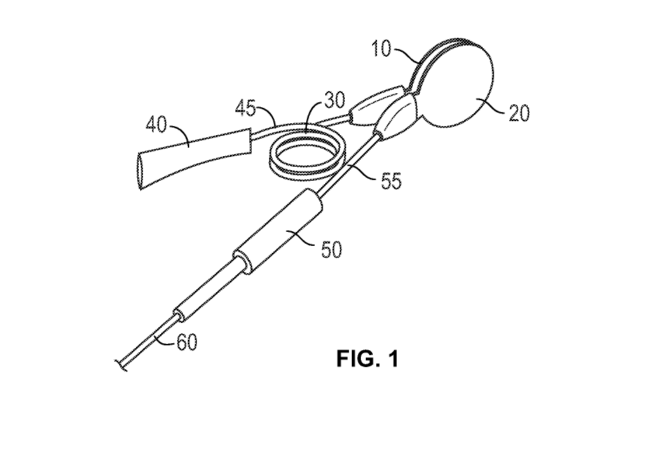

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENTS OF DISCLOSURE

Exemplary embodiments of the methods and apparatuses of the present disclosure will now be described with reference to the figures.

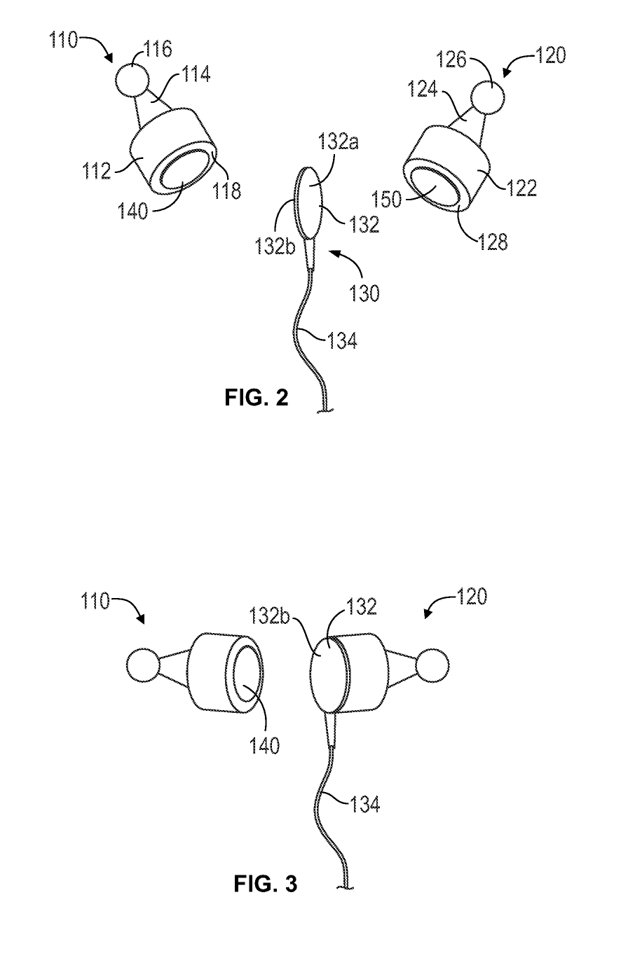

FIG. 2 is an illustration of a biological interface having magnetic attachments and a sensor according to an exemplary embodiment of the present disclosure. A magnetic attachment 110 has a base 112 with a bottom 118 having a magnet 140 having a polarity. The magnetic attachment 110 can have a cone-shaped portion 114 and a dome 116, making it easy to grip and hold using fingers. A magnetic attachment 120 can have a base 122 with a bottom 128, and a cone-shaped portion 124 and a dome 126. The magnetic attachment 120 can have a magnet 150 having a polarity opposite to the polarity of the magnet 140 of magnetic attachment 110 (e.g., north-south polarities). The magnetic attachment 110 can have a cone-shaped portion 114 and a dome 116, making it easy to grip and hold using fingers.

The magnetic attachments 110 and 120 can be made of plastic or any other material, such as metal, wood, or composite material. The magnetic attachments 110 and 120 can be made by injection molding, and the magnets 140 and 150 can be imbedded within the magnetic attachments 110 and 120 in the base 112 and 122, respectively, or can be attached or glued after the injection molding. Various shapes and sizes can be provided for the magnetic attachments 110 and 120, and the exemplary embodiments of the present disclosure are not limited to any particular shapes, colors, sizes or materials. The magnetic attachments 110 and 120 can be manufactured to be lightweight, inexpensive and easy to grip.

A sensor 130 having an electrode 132 connected to a wire 134 can be provided that measures various biological parameters, such as neurological feedback, or recording electrical or other biological activity, or transmitting electrical stimulation. The electrode can have a first surface 132a and a second surface 132b opposite the first surface 132a. The bottom 128 and the magnet 150 can be abraded using a rotary tool to provide a rough surface of the magnet 150 for adhesive attachment to the sensor 130, as will be described below. The first surface 132a of the electrode 132 of the sensor 130 can also be abraded using a rotary tool to provide a roughly prepared surface for adhesion. The bottom 128 of the base 122, the magnet 150 and the first surface 132a of the electrode 132 can be cleaned by, e.g., using rubbing alcohol or other cleaning solution to remove, e.g., oils and loose debris so glue can be applied and firmly adhere.

FIG. 3 is an illustration of a biological interface having magnetic attachments combined with a sensor according to an exemplary embodiment of the present disclosure. Once the bottom 128 of the base 122, the magnet 150 and the first surface 132a of the electrode 132 are dry, glue can be added to the first surface 132a of the electrode 132. The base 128 and magnet 150 are applied on the surface 132a of the electrode 132 to connect the electrode 130 with the magnetic attachment 120. The magnetic attachment 110 can be connected to the electrode 132 on an opposite surface, 132b. Because the magnet 140 of magnetic attachment 110 has a polarity opposite to the magnet 150 of the magnetic attachment 120, the magnetic attachments 110 and 120 will stick together and hold in place while the glue dries. Other methods of attaching the electrode to the magnetic attachment are contemplated by the present disclosure, such as the use of other adhesives, or mechanical mechanisms, and the present disclosure is not limited by any particular mechanism or method.

In use, the magnetic attachment 110 can be placed on one side of a finger, ear lobe or other extremity, and the magnetic attachment 120 having the sensor 130 can be placed on an opposite side of a finger, ear lobe or other extremity. The magnetic attachments are simple to place and are held steady by the magnets 140, 150. The use of the magnets or movement of the magnets does not affect electrical signals being measured by the sensor 130 and provides a closed electronic circuit to measure the signals.

Various other considerations can also be addressed in the exemplary applications described according to the exemplary embodiments of the present disclosure. The exemplary embodiments of the methods and apparatuses described herein can provide, e.g., a biological interface that can reduce placement time by the ease of the use of the magnetic attachments. For example, as magnetic sensors can be placed on opposite sides of, e.g., an ear, ear clips do not need to be placed on two different ears for the same measurements. A ground and a reference can be provided on the same ear or finger rather than each ear requiring preparation and placement of separate ear clips. The magnets do not wear out such as traditional ear clips with wires/coils do. The biological interface of the present disclosure can be used with scalp sensors. The biological interface can have various uses, such as for electroencephalography (EEG) biofeedback, direct current stimulation, transcranial magnetic stimulation, alternating current stimulation, galvanic skin response, electromyography, near infrared spectroscopy, functional near-infrared spectroscopy and electrocardiograms.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure. The disclosures of all documents and publications cited herein are hereby incorporated herein by reference in their entireties.

What is claimed is:

1. A biological interface, comprising:
a first magnetic attachment configured to be placed on a first portion of an extremity of a body, the first magnetic attachment having a first magnet with a first polarity;
a second magnetic attachment configured to be placed on a second portion opposite the first portion of the extremity of the body, the second magnetic attachment having a second magnet with a second polarity opposite to the first polarity; and
a sensor attached to the second magnetic attachment.

2. The biological interface of claim 1, wherein the first magnetic attachment comprises:
a base configured to hold the first magnet within the base of the first magnetic attachment, wherein a bottom of the base exposes a portion of the first magnet.

3. The biological interface of claim 2, further comprising:
a cone shaped portion attached to the base at a first end and a dome at a second end.

4. The biological interface of claim 2, wherein the second magnetic attachment comprises:
a base configured to hold the second magnet within the base of the second magnetic attachment, wherein a bottom of the base exposes a portion of the second magnet.

5. The biological interface of claim 4, further comprising:
a cone shaped portion attached to the base at a first end and a dome at a second end.

6. The biological interface of claim 1, wherein a body of the magnetic attachments are made of plastic.

7. The biological interface of claim 1, wherein the biological interface is used for electroencephalography.

8. The biological interface of claim 1, wherein the sensor comprises an electrode.

9. The biological interface of claim 8, wherein the electrode has a first surface and a second surface opposite the first surface, and the first surface of the electrode is attached to the second magnetic attachment.

10. The biological interface of claim 8, wherein the sensor is glued to the second magnetic attachment.

11. The biological interface of claim 1, wherein the extremity comprises a finger or ear lobe.

12. A method of manufacturing a biological interface, comprising:
providing a first magnet having a first polarity within a first magnetic attachment;
providing a second magnet having a second polarity opposite the first polarity within a second magnetic attachment; and
attaching a sensor to the second magnetic attachment for recording electrical activity.

13. The method of claim 12, wherein the sensor comprises an electrode.

14. The method of claim 13, wherein the electrode has a first surface and a second surface opposite the first surface, and the first surface of the electrode is attached to the second magnetic attachment.

15. The method of claim 14, further comprising:
abrading the first surface of the electrode and a base of the second magnetic attachment before attaching the sensor and the second magnetic attachment.

16. The method of claim 14, wherein the attaching comprises gluing the first surface of the electrode to a base of the second magnetic attachment.

17. The method of claim 12, wherein the first magnetic attachment comprises a base configured to hold the first magnet within the base of the first magnetic attachment, wherein a bottom of the base exposes a portion of the first magnet.

18. The method of claim 17, wherein the second magnetic attachment comprises a base configured to hold the second magnet within the base of the second magnetic attachment, wherein a bottom of the base exposes a portion of the second magnet.
